(19) United States
(12) Patent Application Publication
(10) Pub. No.: US 2020/0161002 A1
(43) Pub. Date: May 21, 2020

(54) PREDICTING AN OCCURRENCE OF A SYMPTOM IN A PATIENT

(71) Applicant: INTERNATIONAL BUSINESS MACHINES CORPORATION, Armonk, NY (US)

(72) Inventors: Chiaki Oishi, YOKOHAMA (JP); Yutaka Oishi, Kawasaki (JP); Takuya Goto, TOKYO (JP); Masaki Saitoh, YOKOHAMA (JP); Shuji Umehara, Kawasaki (JP); Pinaki C. Dey, TOKYO (JP)

(21) Appl. No.: 16/197,442

(22) Filed: Nov. 21, 2018

(51) Int. Cl. G16H 50/50; G06N 20/00

(52) U.S. Cl. CPC G16H 50/50; G06N 20/00

(57) ABSTRACT

A method, computer system, and computer program product for predicting an occurrence of a symptom in a patient are provided. The embodiment may include reading, into a memory, a plurality of time-series prediction models used for predicting the occurrence of the symptom, wherein the time-series prediction models were trained in advance using plural data sets of training data obtained from a plurality of patients, each training data comprising prodrome data and data associated with the occurrence of the symptom. The embodiment may also include selecting at least one time-series prediction model from the time-series prediction models using historical data sets of prodrome data obtained from a patient and data associated with the occurrence of the symptom. The embodiment may further include inputting, to the at least one selected time-series prediction model, current prodrome data obtained from the patient to output a result predicting the occurrence of the symptom.

### BACKGROUND

The present invention relates generally to a prediction technique, and more particularly to predicting an occurrence of a symptom in a patient.

Epilepsy is brain disorders of repeating epileptic seizures and occurs in 0.5 to 1 person in a population of 100 people (0.5 to 1%). Epilepsy most frequently occurs at the age of three or less. After the disorder occurs, treatment continues in many cases. Epilepsy patients are required to be always cautious against an injury due to falling at the time of a seizure, against an accident during driving an automobile, or the like. There are many cases that patients are difficult to continue commutation to and from school or office. As far as the diagnosis is concerned, the diagnosis can be made using electroencephalogram (EEG). The EEG is a painless test which records an electrical activity of a brain. The results of the EEG recordings help doctor diagnosis and make decisions about appropriate treatment.

### SUMMARY

Aspects of the present invention are directed to a method, computer system, and computer program product for adapting a trained neural network having one or more batch normalization layers.

According to an aspect of the present invention, a computer-implemented method for predicting an occurrence of a symptom in a patient. The method comprises reading, into a memory, a plurality of time-series prediction models used for predicting the occurrence of the symptom, wherein the time-series prediction models were trained in advance using plural data sets of training data obtained from a plurality of patients, each training data comprising prodrome data and data associated with the occurrence of the symptom; selecting at least one time-series prediction model from the time-series prediction models using historical data sets of prodrome data obtained from a patient and data associated with the occurrence of the symptom; and inputting, to the at least one selected time-series prediction model, current prodrome data obtained from the patient to output a result predicting the occurrence of the symptom.

According to an aspect of the present invention, a computer system is provided. The computer system may include one or more computer processors, and a memory storing a program which, when executed on the processor, performs an operation for performing the disclosed method.

According to an aspect of the present invention, a computer program product is provided. The computer program product may comprise a computer readable storage medium having program instructions embodied therewith. The program instructions are executable by a computer to cause the computer to perform the disclosed method.

### BRIEF DESCRIPTION OF THE DRAWING

The disclosure will provide details in the following description of preferred embodiments with reference to the following figures. The figures are not necessarily to scale. The figures are merely schematic representations, not intended to portray specific parameters of the invention. The figures are intended to depict only typical embodiments of the invention. In the figures, like numbering represents like elements.

FIG. 1 is a block diagram depicting a computer system used in accordance with an embodiment of the present invention.

FIG. 2 is a flowchart depicting a process of generating a plurality of time-series prediction models used for predicting an occurrence of a symptom.

FIGS. 3A and 3B are flowcharts depicting a process of predicting an occurrence of a symptom in a patient, for example, a specific patient.

FIG. 3C is a flowchart depicting a process of retraining a time-series prediction model.

FIG. 4 is an overall functional block diagram of depicting a computer system hardware in relation to the process of FIGS. 3A and 3B, and optionally FIG. 3C, in accordance with an embodiment of the present invention.

FIG. 5 is an embodiment of the present invention in a case where a disease or an illness is epilepsy.

FIG. 6 depicts a cloud computing environment according to an embodiment of the present invention.

FIG. 7 depicts abstraction model layers according to an embodiment of the present invention.

### DETAILED DESCRIPTION

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As will be appreciated by those of skill in the art, an embodiment of the present invention may be embodied as a method, a computer system, or a computer program product. Accordingly, an embodiment of the present invention may take the form of an entirely hardware-based embodiment, an entirely software-based embodiment, including, for example, firmware, resident software ad micro-code, and the like, or may take the form of an embodiment combining software-based and hardware-based aspects, which may be collectively referred to herein as a “circuit,” a “module,” or a “system”.

As used herein, the expression “a/one” should be understood as “at least one.” The expression “comprise(s)/comprising a/one” should be understood as “comprise(s)/comprising at least one”. The expression “comprise(s)/comprising” should be understood as “comprise(s)/comprising at least”. The expression “/” should be understood as “and/or”.

To define more clearly terms as used herein, exemplified definitions of the terms are provided hereinafter, this should be interpreted broadly as known to a person skilled in the art or a technical field to which the present invention pertains.

As used herein, the term “a symptom” may be any symptom whose occurrence can be predicted in a time-series manner from prodrome data. The term may further refer to a departure from normal function or feeling which is noticed by a patient.

As used herein, the term “a patient” may refer to a person who is suffering from a disease or an illness whose occurrence can be predicted in a time-series manner from a prodrome.

As used herein, the term “a specific patient” may refer to a person of interest who is a subject of an embodiment of the present invention.

As used herein, the term “a disease” may refer to a condition that has established reasons behind it.

As used herein, the term “an illness” may refer to a vague condition that causes discomfort or pain.

The disease may overlap or not overlap with the illness with each other. The disease or the illness may be selected from the group consisting of, for example, but not limited to, epilepsy, convulsive disorder, arrhythmia, myocardial infarction, a panic disorder, hyperventilation syndrome and asthma.

As used herein, the term “prodrome” may refer to any physical or psychological symptom or a set of symptoms that predictably precedes a seizure, which may occur on a scale of days to an hour prior to seizure onset. Prodrome may be restricted to phenomena which do not encompass part of the actual seizure itself. Phenomena commonly seen may be subjective symptoms such as irritability, anxiety and headache, fever, change in feeling and behavior, sleep disorder, slight head heavy feeling, uneasiness, and reduction in concentration. Typically, the prodrome may not be directly related to a disease or an illness.

The prodrome is different from aura in that the aura appears after prodrome. Aura is a seize itself or a complex partial seizure. Aura typically occurs at least one minute before a seize occurs.

For example, in a case where a disease is epilepsy, examples of prodrome and aura may be respectively as follows:

Prodrome: headache; talkativeness, rough tongue; sense of unease (anxiety); irritability; hyperphagia; decline in concentration; changes in mood; depressive mood; derealization; restlessness; and verbosity.

Aura: epigastric sensation; nausea, aphasia; illusion; déjà vu; dreamy state; fear; and visual symptom.

For another example, in a case where a disease is migraine, examples of prodrome and aura may be respectively as follows:

Prodrome: irritability; depression; yawning; increased need to urinate; food cravings; sensitivity to light or sounds; problems in concentrating; fatigue and muscle stiffness; difficulty in speaking and reading; nausea; and difficulty in sleeping.

Aura: visual disturbance; temporary loss of sight; numbness; and tingling on part of the body.

As used herein, the term “training” or “retraining” refers to the process by which a model develops and generates or updating an operating model based on training data, respectively.

As used herein, the term “training data” or “data sets of training data” refers to any data and information input to a model in training. The training data may take the form of, for example, electronic files or records.

The idea of an embodiment of the present invention is based on the following perceptions.

There are existing techniques including a method achieved by integrating multivariable statistical process control (MSPC) and hear rate variability (HRV) analysis. The method focuses on “aura” and predicts a seizure at least one minute before the seizure occurs, thereby allowing a patient to avoid an accident and secure a patient's safety. However, according to the latest seizure prediction, adjustment in consideration of schedules of a job and a trip is difficult for the seizure prediction immediately before the seizure occurs. Capability of detection in an earlier time point is desirable in order to improve the quality of life.

Accordingly, there is a need for detecting a symptom in an earlier time point in order to improve the quality of life.

The inventors focus on prodrome that occurs on a scale of days to an hour prior to seizure onset. Prodrome is a symptom that may include headache, fever, change in feeling and behavior, sleep disorder, slight head heavy feeling, uneasiness, and reduction in concentration. Prodrome can be predicted and detected based on variation in values that can cause these symptoms, such as hormone, metabolism, and autonomic nerves, using a time-series prediction model which is a kind of a machine learning model.

Hereinafter, the various embodiments of the present invention will be described with reference to the accompanying Figures.

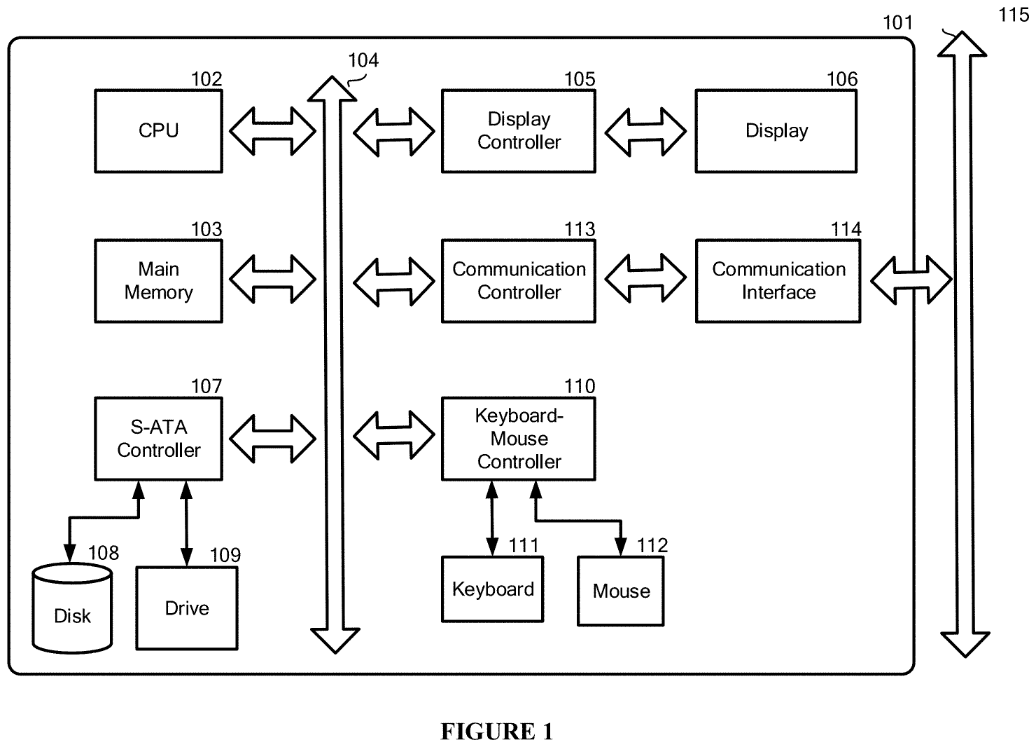

With reference now to FIG. 1, FIG. 1 is an exemplified system architecture of a server computer which may be used in accordance with an embodiment of the present invention. Hereinafter, the term “a server computer” may be also simply referred to as “a server”.

A server 101 may be, for example, but is not limited to, a workstation, a rack-mount type server, a blade type server, a mainframe server, or a cloud server and may run, for example, a hypervisor for creating and running one or more virtual machines. The server 101 may comprise one or more CPUs 102 and a main memory 103 connected to a bus 104. The CPU 102 may be preferably based on a 32-bit or 64-bit architecture. The CPU 102 may be, for example, but is not limited to, the Power® series of International Business Machines Corporation; the Core i™ series, the Core 2™ series, the Atom™ series, the Xeon™ series, the Pentium® series, or the Celeron® series of Intel Corporation; or the Phenom™ series, the Athlon™ series, the Turion™ series, or Sempron™ of Advanced Micro Devices, Inc. (“Power” is registered trademark of International Business Machines Corporation in the United States, other countries, or both; “Core i”, “Core 2”, “Atom”, and “Xeon” are trademarks, and “Pentium” and “Celeron” are registered trademarks of Intel Corporation in the United States, other countries, or both; “Phenom”, “Athlon”, “Turion”, and “Sempron” are trademarks of Advanced Micro Devices, Inc. in the United States, other countries, or both).

A display 106 such as a liquid crystal display (LCD) may be connected to the bus 104 via a display controller 105. The display 106 may be used to display, for management of the computer(s), information on a computer connected to a network via a communication line and information on software running on the computer using an appropriate graphics interface. The display may have a touch screen or a non-touch screen. The display may be for example, but not limited to, a LCD, PDP, OEL or a projection type display. A disk 108 such as a hard disk or a solid-state drive, SSD, and a drive 109 such as a CD, a DVD, or a BD (Blu-ray disk) drive may be connected to the bus 104 via an SATA or IDE controller 107. Moreover, a keyboard 111 and a mouse 112 may be connected to the bus 104 via a keyboard-mouse controller 110 or USB bus (not shown).

An operating system, programs providing Windows®, UNIX® Mac OS®, Linux®, or a Java® processing environment, Java® applications, a Java® virtual machine (VM), and a Java® just-in-time (JIT) compiler, such as J2EE®, other programs, and any data may be stored in the disk 108 to be loadable to the main memory. (“Windows” is a registered trademark of Microsoft corporation in the United States, other countries, or both; “UNIX” is a registered trademark of the Open Group in the United States, other countries, or both; “Mac OS” is a registered trademark of Apple Inc. in the United States, other countries, or both; “Linux” is a registered trademark of Linus Torvalds in the United States, other countries, or both; and “Java” and “J2EE” are registered trademarks of Oracle America, Inc. in the United States, other countries, or both).

The drive 109 may be used to install a program, such as the computer program of an embodiment of the present invention, readable from a CD-ROM, a DVD-ROM, or a BD to the disk 108 or to load any data readable from a CD-ROM, a DVD-ROM, or a BD into the main memory 103 or the disk 108, if necessary.

A communication interface 114 may be based on, for example, but is not limited to, the Ethernet® protocol. The communication interface 114 may be connected to the bus 104 via a communication controller 113, physically connects the server 101 to a communication line 115 and may provide a network interface layer to the TCP/IP communication protocol of a communication function of the operating system of the server 101. In this case, the communication line 115 may be a wired LAN environment or a wireless LAN environment based on wireless LAN connectivity standards, for example, but is not limited to, IEEE® 802.11a/b/g/n (“IEEE” is a registered trademark of Institute of Electrical and Electronics Engineers, Inc. in the United States, other countries, or both).

An embodiment of the present invention comprises the following steps:

A. Preparing a plurality of time-series prediction models;

B. Selection of at least one time-series prediction model from plurality of time-series prediction models;

C. Prediction of an occurrence of a symptom in the patient; and

D. Retraining of the selected time-series prediction model.

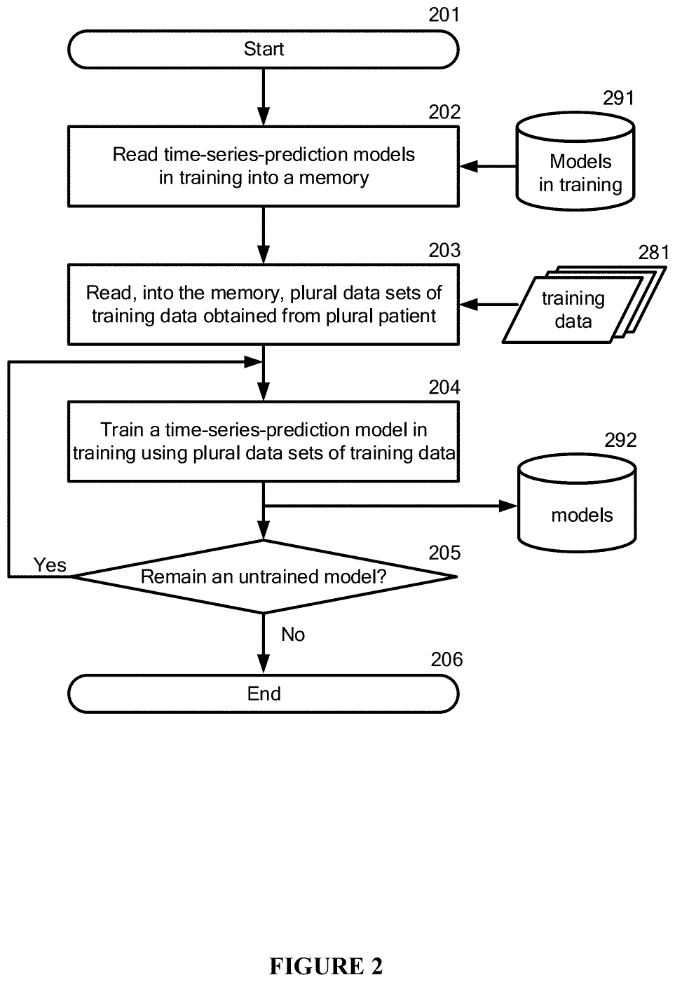

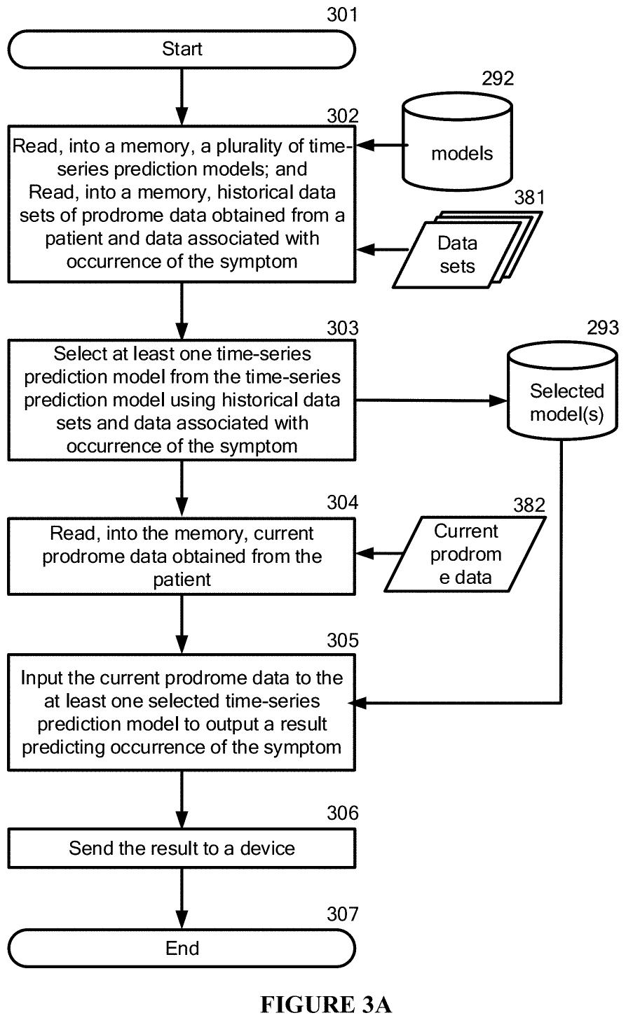

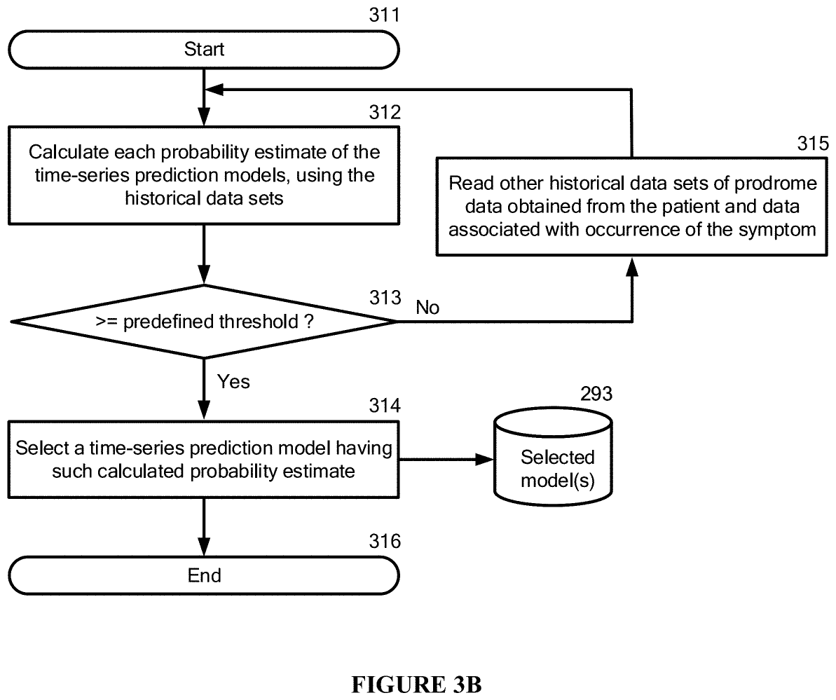

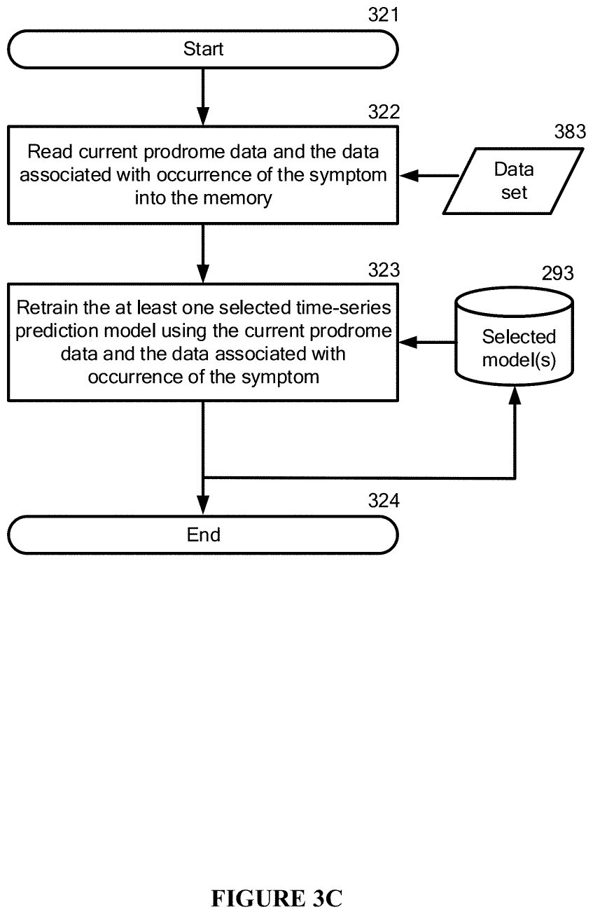

Step A will be explained below by referring to FIG. 2. Steps B and C will be explained below by referring to FIGS. 3A and 3B. Step D will be explained below by referring to FIG. 3C.

With reference now to FIG. 2, FIG. 2 is a flowchart depicting a process of generating a plurality of time-series prediction models used for predicting the occurrence of a symptom.

It is difficult for each patient to obtain time-series data required for training a time-series prediction model, in view of frequency of occurrences of a symptom in a patient. Accordingly, a system that uses known data having high similarity is constructed, and then the accuracy of the system is improved for a respective patient use.

The time-series prediction models may be, for example, but not limited to, Vector Auto Regressive (VAR) model or Long Short-Term Memory (LSTM) model, both of which are well known in the art.

The time-series prediction models are trained in advance using plural data sets of training data obtained from a plurality of patients prior to starting processes described in the flowcharts in FIGS. 3A to 3C.

A subject of each step in FIG. 2 may be the server 101 described in FIG. 1 or a computer which is different from the server 101 and does not carry out an embodiment of the present invention. Hereinafter, let us suppose that the aforesaid subject is the computer for ease of explanation.

At step 201, the computer starts the aforesaid process.

At step 202, the computer reads, into a memory, a plurality of time-series prediction models in training from a storage 291 which can be accessible by the computer. The time-series prediction models can be used for predicting the occurrence of a symptom by inputting current prodrome data obtained from a patient.

At step 203, the computer reads, into the memory, plural data sets of training data 281 obtained from a plurality of patients. Different data sets of training data can be provided for each of the plurality of time-series prediction models. The plurality of patients from which the training data was collected may suffer from the same or similar disease or illness. Each training data comprises prodrome data and data associated with the occurrence of the symptom, both of which were obtained from the same patient in the plurality of patients.

The prodrome data may comprise data relating to prodrome. As stated above, the prodrome may comprise any physical or psychological symptom or a set of symptoms that predictably precedes a seizure, which may occur on a scale of days to an hour prior to seizure onset.

In one embodiment of the present invention, the prodrome data may comprise at least behavior data obtained from the patient.

The behavior data may be any data from which prodrome can be detected. The behavior data may be, for example, but not limited to, data relating to a movement of a line of sight of the patient operating a mobile device, data relating to an activity condition of the patient operating a mobile device, data relating to a typographical mistake or typo made by the patient operating a mobile device, or a combination thereof. The activity condition may comprise slow or fast condition or condition comprising abnormal.

The behavior data may be, for example but not limited to, image data or video data. The image data or video data was taken by, for example but not limited to, a digital or video camera.

The prodrome data may comprise data obtained from a mobile device attached to or had by the patient or data obtained from a digital or video camera equipped in a room.

The prodrome data may comprise photographed data of the patient. The photographed data may be obtained from a digital or video camera capable of photographing the patient.

The prodrome may not be directly related to a disease of the patient. The data associated with the occurrence of the symptom comprises data at the time when a symptom really occurred after such prodrome was observed.

The data associated with the occurrence of the symptom may comprise, for example, but not limited to, starting time at the time when the symptom occurs, ending time of the symptom, information on the symptom, or a combination thereof.

The data associated with the occurrence of the symptom may be epilepsy seizure data if a disease or an illness is epilepsy.

The training data on each patient may be obtained in a freely-selected time period.

Steps 202 and 203 may be carried out simultaneously or inversely.

At step 204, the computer trains a time-series-prediction model in training using the plural data sets of training data. After the training, the computer stores the trained time-series-prediction model into a storage 292.

At step 205, the computer judges whether there remains an untrained model or not. If the judgment is positive, the computer proceeds back to 204 in order to train the untrained model. If the judgment is negative, the computer proceeds to step 206.

At step 206, the computer terminates the aforesaid process.

The trained time-series-prediction model can be thereafter used in an embodiment of the present invention of predicting an occurrence of a symptom in a patient, for example, a specific patient.

FIGS. 3A and 3B are flowcharts depicting a process of predicting an occurrence of a symptom in a patient, for example, a specific patient.

A subject of each step in FIGS. 3A and 3B may be a computer system such as the server 101 described in FIG. 1. Hereinafter, let us suppose that the aforesaid subject is the computer system.

With reference now to FIG. 3A, FIG. 3A is a flowchart of a basic process of predicting an occurrence of a symptom in a patient.

At step 301, the computer system starts the aforesaid process.

At step 302, the computer system reads, into a memory, a plurality of time-series prediction models from the storage 292. The computer system further reads, into the memory, historical data sets 381 of prodrome data obtained from a patient and data associated with the occurrence of the symptom.

At step 303, the computer system selects at least one time-series prediction model from the time-series prediction model using historical data sets of prodrome data obtained from a patient and data associated with the occurrence of the symptom. The computer system may store the selected time-series prediction model into a storage 293. The detailed embodiment of step 303 will be explained below by referring to FIG. 3B.

At step 304, the computer system reads, into the memory, current prodrome data 382 obtained from the patient.

At step 305, the computer system may read the at least one selected time-series prediction model from the storage 293, if necessary. The computer system then inputs the current prodrome data to the at least one selected time-series prediction model to output a result predicting the occurrence of the symptom.

At step 306, the computer system sends the result to a device associated with the patient, a family of the patient, or a medical doctor or counselor in health guide who consults with the patient. The device may be, for example, but not limited to a mobile device such as a smartphone, a mobile phone, a tablet, a book reader; a wearable device such as a smart watch, a smart glove and a smart glasses; and a personal computer such as a note book computer. In response to receiving the result, the device may display or announces the result.

At step 307, the computer terminates the aforesaid process.

In response to displaying or announcing the result, the patient, the family of the patient, or the medical doctor or counselor of the patient can take any necessary or appropriate action in advance before a symptom really occurs. The action may comprise suspending operations, suspending driving, taking a medicine or going to the hospital.

After the symptom really occurs in the patient, the patient can input the data relating to the really occurred symptom to the device or the device can automatically generate the data relating to the really occurred symptom. The device may generate new data associated with the occurrence of the symptom from the data relating to the really occurred symptom and then send the new data to the computer system. In response to receiving the new data associated with the occurrence of the symptom, the computer system stores, as a set of retraining data, the current prodrome data and the new data associated with the occurrence of the symptom. Thus, the set of retraining data comprises the current prodrome data and the new data associated with the occurrence of the symptom.

The set of retraining data will be used for retraining the selected at least one time-series prediction model, as mentioned below by referring to FIG. 3C.

With reference now to FIG. 3B, FIG. 3B is a flowchart of a detailed process.

At step 311, the computer system starts the aforesaid process in response to starting of step 303 described in FIG. 3A.

At step 312, the computer system calculates each probability estimate of the time-series prediction models, using the historical data sets. The calculation can be made using, for example but not limited to, a technique of Bayesian model or portfolio, both of which are well-known in the art.

At step 313, the computer system judges whether the calculated probability estimate is equal or above a predefined threshold or not. If the judgment is positive, the computer system proceeds to step 314. Meanwhile, if the judgment is negative, the computer system proceeds to step 315.

At step 314, in response to the positive judgment, the computer system selects a time-series prediction model having such calculated probability estimate. The computer system may store the selected time-series prediction model into the storage 293.

At step 315, in response to the negative judgment, the computer system reads other historical data sets of prodrome data obtained from the patient and data associated with the occurrence of the symptom. The computer system then proceeds back to step 312 in order to recalculate each probability estimate of the time-series prediction models using the other historical data sets.

At step 316, the computer system terminates the aforesaid process and then goes back to step 304 described in FIG. 3A.

With reference now to FIG. 3C, FIG. 3C is a flowchart depicting a process of retraining a time-series prediction model.

After the process described in FIG. 3A is terminated, the selected model is trained in order to improve accuracy of a result predicting the occurrence of the symptom.

A subject of each step in FIG. 3C may be a computer system such as the server 101 described in FIG. 1 or a computer which is different from the server 101 and does not carry out an embodiment of the present invention. Hereinafter, let us suppose that the aforesaid subject is the computer for ease of explanation.

At step 321, the computer system starts the aforesaid process after carrying out step 306 described in FIG. 3A and further occurring real occurrence of the symptom.

At step 322, the computer system reads the current prodrome data described in step 305 of FIG. 3A and the data associated with the occurrence of the symptom 383 into the memory.

At step 323, the computer system may read, into the memory, the selected model from the storage 293, if necessary. The selected model is a subject to be retrained. The computer system then retrains the at least one selected time-series prediction model using the current prodrome data and the data associated with the occurrence of the symptom. The computer system may store the retrained time-series prediction model into the storage 293 and therefore the time-series prediction model before retraining is replaced with the retrained time-series prediction model.

At step 324, the computer system terminates the aforesaid process.

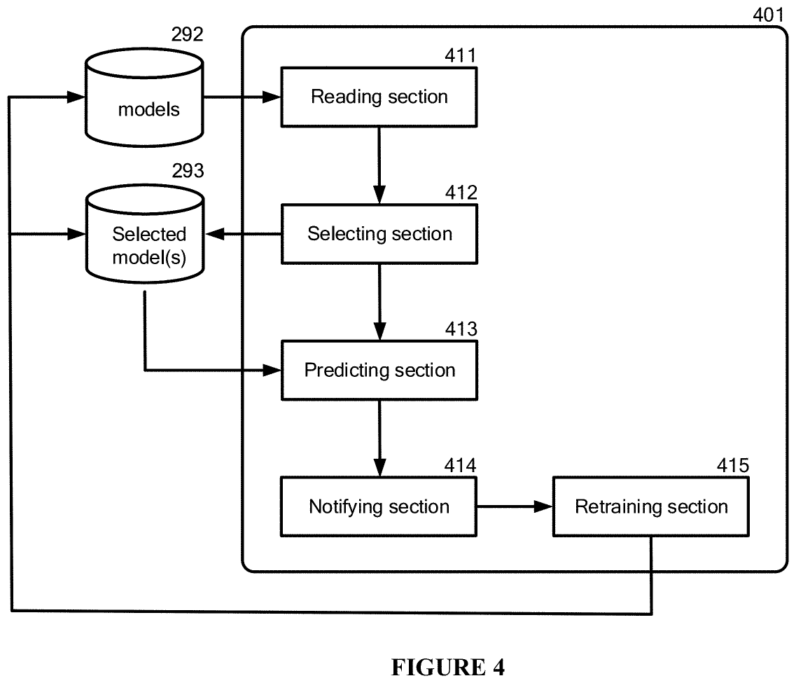

With reference now to FIG. 4, FIG. 4 is an overall functional block diagram of depicting a computer system hardware in relation to the process of FIGS. 3A and 3B, and optionally FIG. 3C, in accordance with an embodiment of the present invention.

A computer system 401 may correspond to the server 101 described in FIG. 1.

The computer system 401 may comprise a reading section 411, a selecting section 412, a predicting section 413, and a notifying section 415. The computer system 401 may further comprise a retraining section 415.

The reading section 411 reads, into a memory, a plurality of time-series prediction models used for predicting an occurrence of a symptom.

The reading section 411 may perform step 302 described in FIG. 3A.

The selecting section 412 selects at least one time-series prediction model from the time-series prediction models using historical data sets of prodrome data obtained from a patient and data associated with the occurrence of the symptom. The selecting section 412 may calculate each probability estimate of the time-series prediction models, using the historical data sets. The selecting section 412 may select a time-series prediction model having such calculated probability estimate if the calculated probability estimate is equal or above a predefined threshold. Meanwhile if the calculated probability estimate is below a predefined threshold, the selecting section 412 may use other historical data sets of prodrome data obtained from the patient and data associated with the occurrence of the symptom for recalculating each probability estimate of the time-series prediction models using the other historical data sets.

The selecting section 412 may perform step 303 described in FIG. 3A and steps 312 to 315 described in FIG. 3B.

The predicting section 413 inputs, to the at least one selected time-series prediction model, current prodrome data obtained from the patient to output a result predicting the occurrence of the symptom.

The predicting section 413 may perform steps 304 to 306 described in FIG. 3A.

The notifying section 414 may send the result to a device associated with the patient, a family of the patient, or a medical doctor or counselor in health guide who consults with the patient.

The notifying section 414 may perform step 306 described in FIG. 3A.

The retraining section 415 may retrain the at least one selected time-series prediction model using the current prodrome data and the data associated with the occurrence of the symptom.

The retraining section 415 may perform steps 322 and 323 described in FIG. 3C.

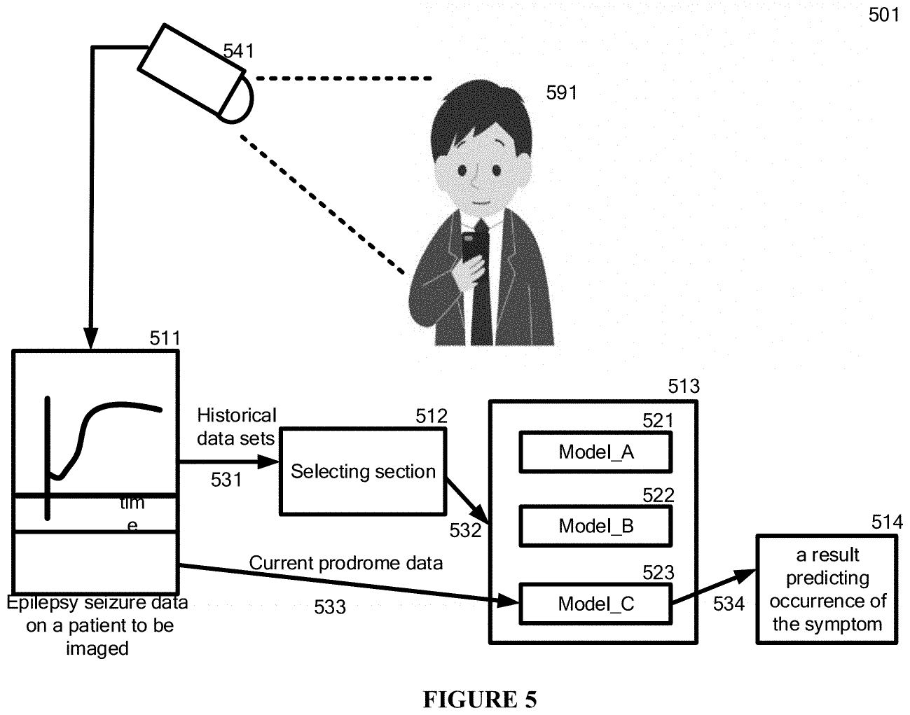

With reference now to FIG. 5, FIG. 5 is an embodiment of the present invention in a case where a disease or an illness is epilepsy.

### 1. Preparing Time-Series Prediction Models A to C

Let us suppose that time-series prediction models A to C in training were trained in advance using plural data sets of training data obtained from a plurality of patients prior to starting a process for predicting an occurrence of a symptom in the patient 591. The trained time-series prediction models A to C 521, 522 and 523 were stored in a storage 513.

The training data comprise prodrome data and data associated with an occurrence of a symptom.

The prodrome data comprises at least behavior data obtained from the patients. The behavior data was generated by taking one patient using video camera and processing the taken video data as the behavior data. The video data includes movements of the aforesaid patient.

The behavior data comprises data relating to a movement of a line of sight of the patients operating a mobile device, data relating to an activity condition of the patients operating a mobile device, data relating to a typographical mistake or typo made by the patients operating a mobile device, or combination of these.

The data associated with the occurrence of the symptom comprises at least starting time at the time when the symptom occurs, ending time of the symptom, information on the symptom, or a combination thereof.

### 2. Selection of at Least One time-Series Prediction Model from the Trained Time-Series Prediction Models A to C 521, 522 and 523

Let us suppose that the patient 591 suffers from epilepsy and is a subject to which an embodiment of the present invention is applied.

The computer system reads 531 historical data sets of prodrome data obtained from a patient and data associated with the occurrence of the symptom.

The prodrome data in the historical data sets comprise at least behavior data obtained from the patients, correspond to the prodrome data in the training data.

The data associated with the occurrence of the symptom in the historical data sets comprise at least starting time at the time when the symptom occurs, ending time of the symptom, information on the symptom, or a combination thereof, corresponding to the data associated with the occurrence of the symptom in the training data.

The computer system then selects 532 at least one time-series prediction model from the time-series prediction models A to C 521, 522 and 523 using the historical data sets of the prodrome data and the data associated with the occurrence of the symptom. The selection can be carried out using a selection section 512 corresponding to the selecting section 412 described in FIG. 4.

Let us suppose that the time-series prediction model C 523 was selected for the patient 591.

### 3. Prediction of an Occurrence of a Symptom in the Patient 591

The computer system receives, from the video camera 541, video data 511 including movements of the patient 591. The video data may be epilepsy seizure data on a patient to be imaged.

The computer system analyzes the video data 511 and then generates the current prodrome.

The computer system inputs 533 the current prodrome data to the selected time-series prediction model C 523.

The computer system then outputs 534 a result predicting the occurrence of the symptom and then store the result in a storage 514.

The computer system may display or announce the result on a mobile device associated with the patient 591, a family of the patient 591, or a medical doctor or counselor in health guide who consults with the patient 591.

In response to displaying or announcing the result, the patient 591, the family of the patient, or the medical doctor or counselor of the patient can take any necessary or appropriate action in advance before a symptom really occurs.

According to this embodiment of the present invention, the quality of life of the patient 591 can be improved.

### 4. Retraining of the Selected Time-Series Prediction Model C 523

After the symptom really occurs in the patient 591, the device may generate new data associated with the occurrence of the symptom and then send it to the computer system. In response to receiving the new data associated with the occurrence of the symptom, the computer system stores, as a set of retraining data, the current prodrome data and the new data associated with the occurrence of the symptom. Thus, the set of retraining data comprises the current prodrome data and the new data associated with the occurrence of the symptom.

The computer system retrains the selected time-series prediction model C 523 using the set of retraining data and then replace the present time-series prediction model C in the storage 513 with the retrained time-series prediction model C.

The present invention may be a method, a system, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

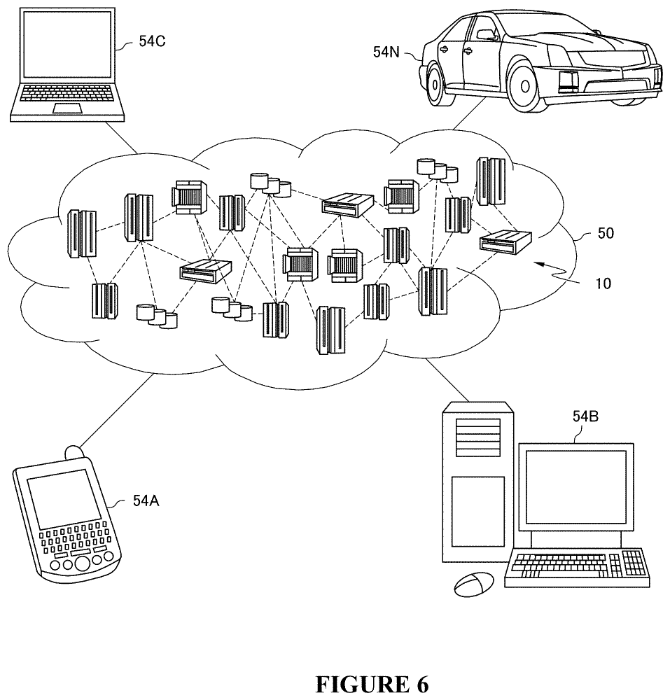

Referring now to FIG. 6, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

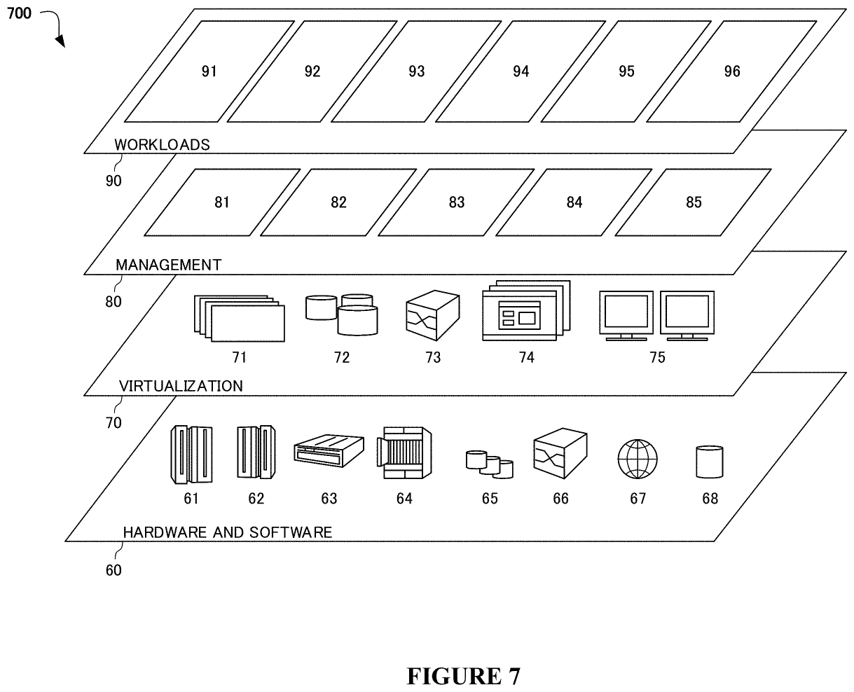

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and navigation processing 96.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Improvements and modifications can be made to the foregoing without departing from the scope of the present invention.

What is claimed is:

1. A computer-implemented method for predicting an occurrence of a symptom in a patient, the method comprising:
reading, into a memory, a plurality of time-series prediction models used for predicting the occurrence of the symptom, wherein the time-series prediction models were trained in advance using plural data sets of training data obtained from a plurality of patients, each training data comprising prodrome data and data associated with the occurrence of the symptom;
selecting at least one time-series prediction model from the time-series prediction models using historical data sets of prodrome data obtained from a patient and data associated with the occurrence of the symptom; and
inputting, to the at least one selected time-series prediction model, current prodrome data obtained from the patient to output a result predicting the occurrence of the symptom.

2. The method according to claim 1, wherein the current prodrome data and data associated with the occurrence of the symptom are used for retraining the at least one selected time-series prediction model.

3. The method according to claim 1, wherein selecting the at least one time-series prediction model comprises calculating each probability estimate of the time-series prediction models, using the historical data sets.

4. The method according to claim 3, wherein, if the calculated probability estimate is equal or above a predefined threshold, a time-series prediction model having the calculated probability estimate is selected.

5. The method according to claim 3, wherein, if the calculated probability estimate is below a predefined threshold, other historical data sets of prodrome data obtained from the patient and data associated with the occurrence of the symptom are used, and each probability estimate of the time-series prediction models is recalculated using the other historical data sets.

6. The method according to claim 1, wherein the prodrome data comprises at least behavior data obtained from the patient.

7. The method according to claim 6, wherein the behavior data is image data or video data.

8. The method according to claim 7, wherein the image data or video data was taken by a digital camera or a video camera.

9. The method according to claim 7, wherein the behavior data is selected from a group consisting of data relating to a movement of a line of sight of the patient operating a mobile device, data relating to an activity condition of the patient operating a mobile device, and data relating to a typographical mistake made by the patient operating a mobile device.

10. The method according to claim 1, wherein the prodrome data comprises data obtained from a mobile device attached to or held by the patient.

11. The method according to claim 1, wherein the prodrome data comprises photographed data of the patient.

12. The method according to claim 1, wherein the symptom is any symptom whose occurrence can be predicted in a time-series manner from prodrome data.

13. The method according to claim 1, wherein prodrome data is not directly related to a disease or an illness of the patient.

14. The method according to claim 13, wherein the disease or the illness is selected from the group consisting of epilepsy, convulsive disorder, arrhythmia, myocardial infarction, a panic disorder, hyperventilation syndrome and asthma.

15. A computer system, comprising:
one or more processors; and
a memory storing a program which, when executed on the processor, performs an operation of predicting an occurrence of a symptom in a patient, the operation comprising:
reading, into a memory, a plurality of time-series prediction models used for predicting the occurrence of the symptom, wherein the time-series prediction models were trained in advance using plural data sets of training data obtained from a plurality of patients, each training data comprising prodrome data and data associated with the occurrence of the symptom;
selecting at least one time-series prediction model from the time-series prediction models using historical data sets of prodrome data obtained from a patient and data associated with the occurrence of the symptom; and
inputting, to the at least one selected time-series prediction model, current prodrome data obtained from the patient to output a result predicting the occurrence of the symptom.

16. The computer system according to claim 15, wherein the current prodrome data and the data associated with the occurrence of the symptom are used for retraining the at least one selected time-series prediction model.

17. The computer system according to claim 15, wherein selecting the at least one time-series prediction model comprises calculating each probability estimate of the time-series prediction models, using the historical data sets.

18. A computer program product for predicting an occurrence of a symptom in a patient, the computer program product comprising a computer readable storage medium having program instructions embodied therewith, the program instructions executable by a computer to cause the computer to perform a method comprising:
reading, into a memory, a plurality of time-series prediction models used for predicting the occurrence of the symptom, wherein the time-series prediction models were trained in advance using plural data sets of training data obtained from a plurality of patients, each training data comprising prodrome data and data associated with the occurrence of the symptom;
selecting at least one time-series prediction model from the time-series prediction models using historical data sets of prodrome data obtained from a patient and data associated with the occurrence of the symptom; and
inputting, to the at least one selected time-series prediction model, current prodrome data obtained from the patient to output a result predicting the occurrence of the symptom.

19. The computer program product according to claim 18, wherein the current prodrome data and the data associated with the occurrence of the symptom are used for retraining the at least one selected time-series prediction model.

20. The computer program product according to claim 18, wherein selecting the at least one time-series prediction model comprises calculating each probability estimate of the time-series prediction models, using the historical data sets.
